(19) United States
(12) Patent Application Publication
(10) Pub. No.: US 2008/0249469 A1
(43) Pub. Date: Oct. 9, 2008

(54) METHOD AND APPARATUS FOR ACTIVE CONTROL OF DRUG DELIVERY USING ELECTRO-OSMOTIC FLOW CONTROL

(76) Inventors: Ponnambalam Selvaganapathy, (US); Geetha Mahadevan, (US); Heather Sheardown, Nobleton (CA)

Correspondence Address: BERESKIN AND PARR, 40 KING STREET WEST, BOX 401, TORONTO, ON, M5H 3Y2, CA

(21) Appl. No.: 12/054,024

(22) Filed: Mar. 24, 2008

Related U.S. Application Data
(60) Provisional application No. 60/896,428, filed on Mar. 22, 2007.
(60) Provisional application No. 60/916,961, filed on May 9, 2007.

(51) Int. Cl. A61M 1/00

(52) U.S. Cl. 604/151

(57) ABSTRACT

A substance delivery apparatus is disclosed. Embodiments of the substance delivery apparatus comprise a housing defining a reservoir containing the substance. At least one micro-needle is operably connected to the reservoir. A micro-pump is fluidically connected to the reservoir so that when the micro-pump is activated, the substance is directed from the reservoir, through the at least one micro-needle.

This application claims the benefit of U.S. Provisional Application No. 60/896,428 filed Mar. 22, 2007 and U.S. Provisional Application No. 60/916,961 filed May 9, 2007, the entire contents of which is hereby incorporated by reference.

The section headings used herein are for organizational purposes only and are not to be construed as limiting the subject matter described in any way.

### FIELD

Applicants' teachings are related to a method, apparatus and use of an apparatus for active control of drug delivery using electro-osmotic flow control. Moreover, the applicants' teachings are directed towards a method, apparatus and use of an apparatus as a controlled delivery vehicle of a drug or substance to, for example, but not limited to, the posterior of an eye. Applicants' teachings are also related to a micro-fluidic pump. Further, applicants' teachings are related to a method of manufacturing micro-needles for use in, for example, but not limited to, a drug delivery apparatus.

### BRIEF DESCRIPTION OF THE DRAWINGS

Exemplary embodiments of the invention are described in further detail below, by way of example only, with reference to the accompanying drawings, in which:

FIG. 1A is a perspective view of a delivery apparatus according to an embodiment of the invention;

FIG. 1B is a is an exploded perspective view of the delivery apparatus of FIG. 1A, showing the interior of the delivery apparatus;

FIG. 2 is a partial cross section taken along line 2-2 in FIG. 1A, showing a reservoir of the delivery apparatus;

FIG. 3 shows the cross-section of FIG. 2, with capillaries inserted;

FIG. 4 is a partial cross-section taken along line 4-4 in FIG. 1A, showing a main chamber of the micro-pump of the delivery apparatus;

FIG. 5 shows the cross-section of FIG. 4, further showing a semi-permeable membrane, and a source of the micropump;

FIG. 6 is a cross section taken along line 6-6 in FIG. 1A;

FIG. 7 shows the cross section of FIG. 6, with sharpened capillaries;

FIG. 8 shows the cross section of FIG. 7, after the substance to be delivered has been loaded; and

FIG. 9 shows the cross section of FIG. 8, after the pump has been activated.

### DESCRIPTION OF VARIOUS EMBODIMENTS

Diseases of the eye, such as age related macular degeneration (AMD), can lead to vision loss. While a variety of new pharmaceuticals have been developed for the treatment of eye diseases, such as age related macular degeneration, the administration of these pharmaceuticals generally involves regular injections into the back of the eye which can be inconvenient and painful for the patient. Risks associated with these injections can include retinal detachment, hemorrhage, endophthalmitis and cataracts.

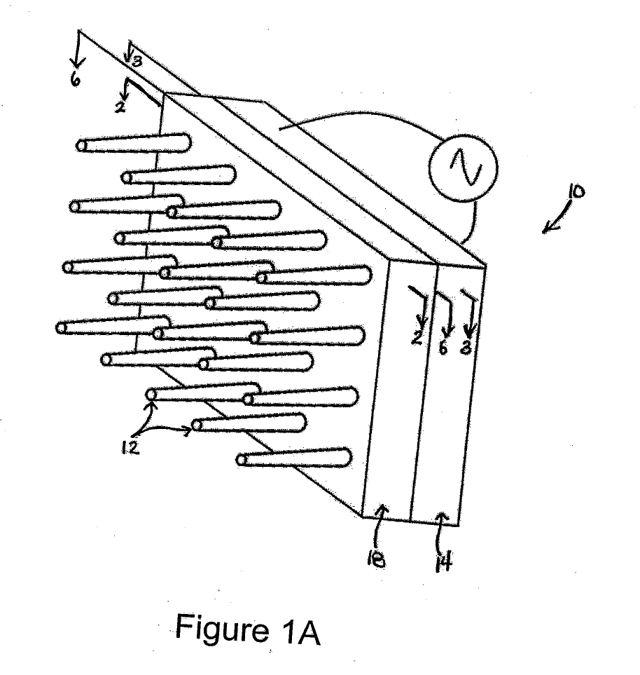

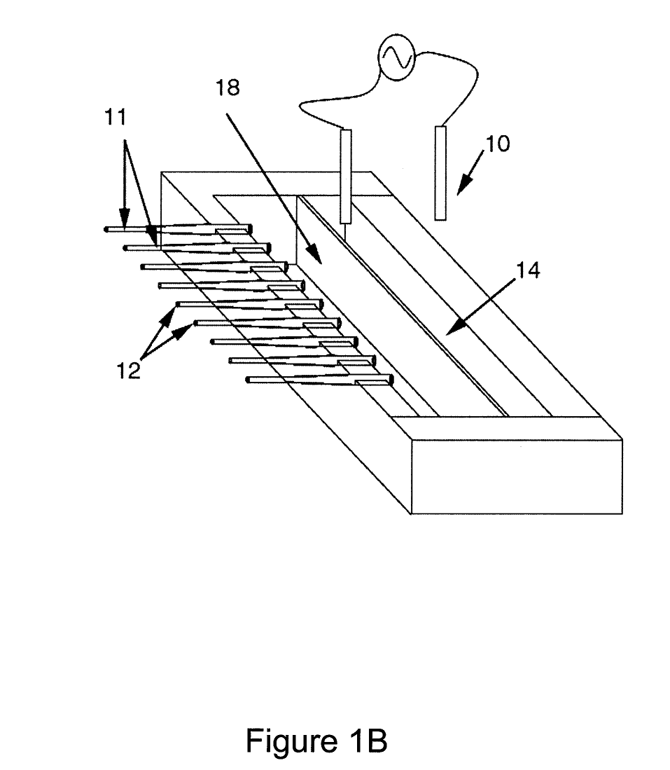

FIGS. 1A and 1B are illustrations of some embodiments of applicants' teachings showing a delivery apparatus 10 that can be used for active control of drug delivery using electro-osmotic flow control. Delivery apparatus 10 comprises micro-needles 12 and a micro-fluidic pump 14. A source 16 to produce a zero average current, such as a symmetrical AC current, is also provided. Delivery apparatus 10 of applicants' teachings is suitable for use as a controlled delivery vehicle of a drug or substance to a targeted area, and generally a tissue, such as, for example, but not limited to, the posterior of an eye. For example, the delivery apparatus 10 may be placed on the external eye and positioned such that it sits posterior to the lens and iris. In this example, the micro-needles 12 penetrate the ocular tissue. Moreover, delivery apparatus 10 of applicants' teachings is suitable for use as a controlled delivery vehicle of a drug over long periods of time. The delivery apparatus 10 can, however, be used in other applications, including transdermal applications.

Delivery apparatus 10 may be of a variety of sizes, depending on the particular application. In some embodiments, delivery apparatus 10 may be up to 10 cm×10 cm×5 mm in size. In some particular embodiments, wherein delivery apparatus 10 is used on the posterior of an eye, delivery apparatus 10 may be 1 cm×1 cm×1 mm in size. In other embodiments, delivery apparatus 10 may be another size, and the invention is not limited in this regard.

In some embodiments of applicants' teachings, the micro-needles 12 are manufactured to be relatively thin and short so that their interaction with the nerves in the tissue of the targeted area, such as the posterior of the eye, is minimized, but allow the transport of drug to the targeted area to be effected by the micro-fluidic pump 14. While the micro-needles 12 shown in FIG. 1 are out of plane needles, in-plane needles may also be used.

The delivery apparatus 10 according to the various embodiments of applicant's teachings, and as illustrated in FIG. 1B, has the micro-needles 12 operably connected to a reservoir 18 containing a substance, such as a drug, to be delivered to the targeted area. The reservoir 18, according to various embodiments of applicants' teachings, is operably connected to micro-fluidic pump 14 so that, when the pump is activated the substance in the reservoir 18 is directed from the reservoir and through the micro-needles 12 to the targeted area. According to some embodiments of applicants' teachings, the micro-fluidic pump 14 and the reservoir 18 are manufactured separately, but operably linked, however other embodiments of applicants' teachings can have the micro-fluidic pump 14 and the reservoir manufactured as an integral construction. Moreover, according to some embodiments of applicants' teaching the delivery of the substance to the targeted area is at a controlled rate, as will hereinafter be explained in greater detail.

In some embodiments of applicants' teachings, the structural material used in construction of the reservoir 18 and the micro-fluidic pump 14 is biocompatible. One example of such a material suitable for use with applicants' teachings is polydimethyl siloxane (PDMS), which is a flexible biocompatible elastomer. Other suitable materials are intended to be covered, however, such as, for example, including polyurethanes, ethylene vinyl acetate, and applicants' teachings are not intended to be limited to PDMS. In alternate embodiments, the structural material used in construction of the reservoir 18 and the micro-fluid pump 14 may not be biocompatible. In such embodiments, the reservoir 18 and the micro-fluid pump 14 may be coated with a biocompatible material.

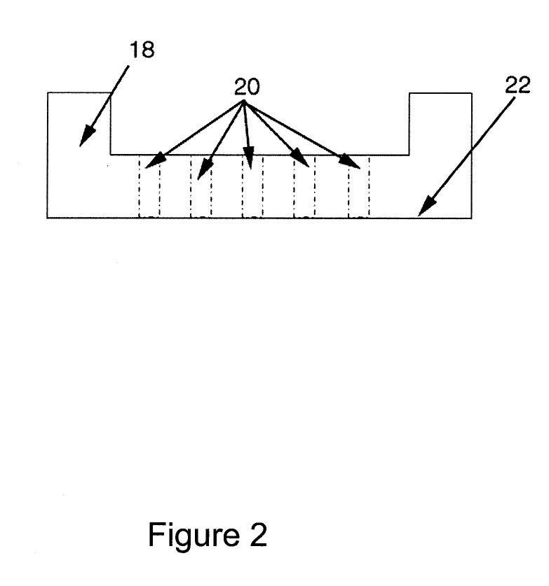

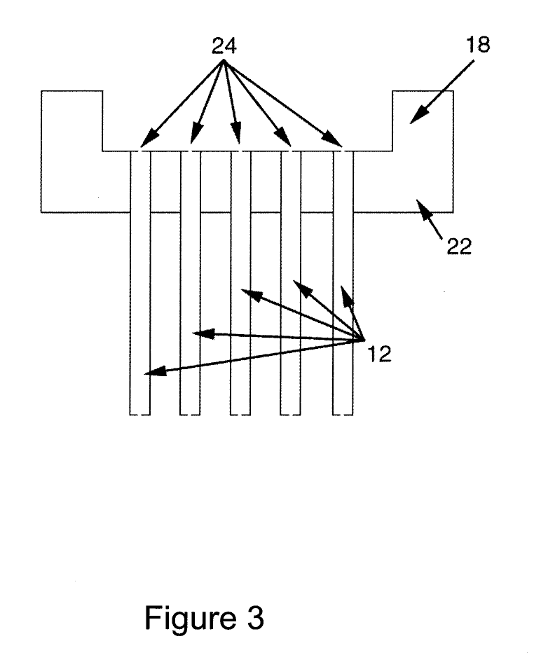

Referring to FIG. 2, the reservoir 18 has, in accordance with certain embodiments of applicants' teachings grooves or channels 20. Channels 20 are shaped to receive one end 24 of micro-needles 12, as illustrated in FIG. 3. Moreover, in accordance with some embodiments of applicants' teachings the channels 20 are spaced along one facing 22 of the reservoir 18 so that the micro-needles, when received therein, are aligned in position along facing 22.

The reservoir 18, according to certain embodiments of applicants' teachings, can be manufactured by creating a mold to cast the PDMS to form the reservoir. The mold can be constructed by, for example, but not limited to multilayer photolithography processes, (X-ray) lithography, electroplating and molding (LIGA), electroforming, electro-discharge machining, focused ion beam machining, and laser machining.

In one illustrative example of applicants' teachings, silicon wafers were spin coated with one hundred micron-thick SU8 photoresist and were subsequently exposed using UV-photolithography for pattern transfer to create the structure of a micro-fluidic network. PDMS prepolymer was cast into this master mold to create replicas of the micro-fluidic network comprising of 300-micron channels 20 spaced apart from one another as illustrated in FIG. 2.

Micro-needles 12 can be shaped, in accordance with some embodiments of applicants' teachings by, for example, but not limited to, techniques including using sacrificial boundary etching and withdrawal control technique. In one illustrative example of applicants' teachings glass micro-needles were fabricated from capillary tubes using a pipette puller to locally melt the glass and pull it to obtain a sharp tip. Continuing the illustrative example, the micro-needles 12 are then subject to plasma oxidation to increase adhesion of end 24 of the micro-needles 12 within the channels 20 of the reservoir 18. Illustrative of applicants' teachings, but not limiting, the micro-needles can have a width of 50 μm-1 mm.

Once the micro-needles 12 and reservoir 18 are assembled, in accordance with some embodiments of applicants' teachings, micro-needles 12 are shaped simultaneously by attaching the delivery apparatus 10 to a micro-positioner and extracting the tip from an etchant solution at a controlled rate, called the controlled withdrawal technique. Using the controlled withdrawal technique, micro-needles 12 are fabricated and shaped individually and then mounted and aligned in the appropriate device.

For some embodiments of applicants' teachings, on the other hand, unshaped capillaries are first assembled and aligned into channels 20 in the reservoir 18, as shown in FIG. 3. The array of micro-needles 12 are then shaped simultaneously by attaching the delivery apparatus 10 to a micro-positioner and extracting the tip from an etchant solution at a controlled rate. The micro-needles 12 can also be shaped, in accordance with some embodiments of applicants' teachings, after assembly of the micro-needles 12 and reservoir 18 with the micro-fluidic pump 14, as will hereinafter be described.

For some embodiments of applicants' teachings etchant solutions include acid solutions such as, for example, hydrofluoric acid. The concentration of the solution and the rate of withdrawal from the etchant solution will define the taper of the micro-needles. Illustrative, but not limiting, needle tip diameters range from 200 nm to 10 μm as measured using a SEM.

The taper of the micro-needles should be sufficient to allow the delivery apparatus 10 to be placed onto the targeted area, such as, for purposes of testing the apparatus, and not to be limited to such an area, the sclera of an enucleated bovine eye and inserted through the vitreous for infusion of a substance, such as a dye, for purposes of testing, into the posterior segment of the eye.

Once the reservoir 18 and micro-needles 12 are assembled, micro-needles 12 may be sealed to reservoir 18. For example, PDMS may be used as an adhesive to seal micro-needles 12 to reservoir 18.

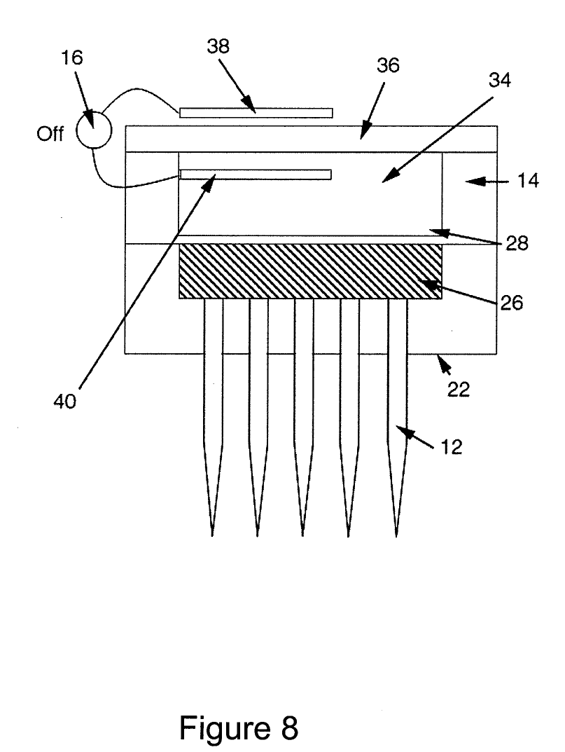

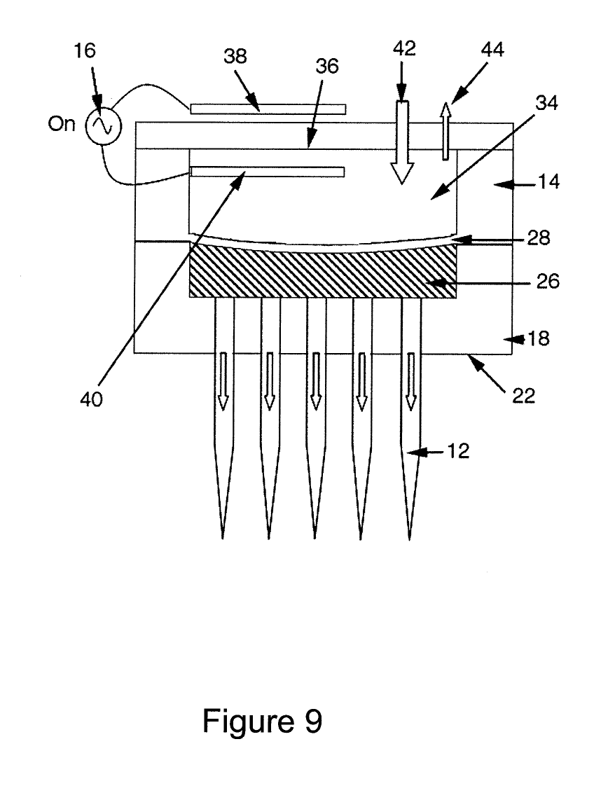

Once the reservoir 18 and micro-needles 12 are assembled, a substance to be delivered, such as drug 26 (see FIGS. 8 and 9), is introduced to the reservoir 18 and sealed in place. The amount of substance introduced into the reservoir 18 may vary depending on the particular application. In some embodiments, between about 10 μL and 100 μL may be introduced into the reservoir 18.

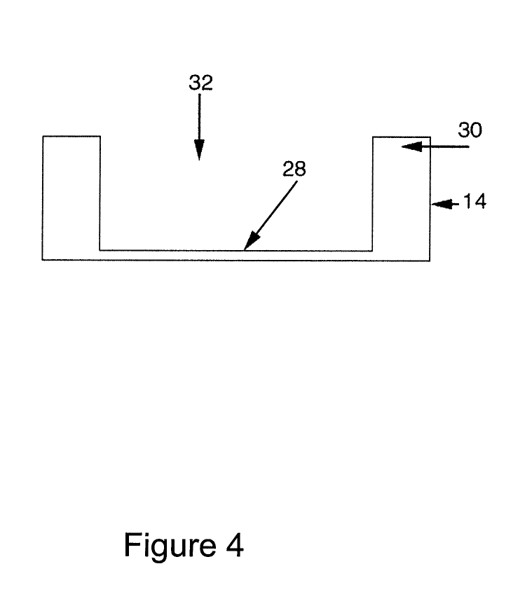

The device 10 may be a single-use device or the reservoir 18 may be reloadable. One illustrative example is to seal the drug 26 in place at room temperature with a thin flexible PDMS diaphragm 28. The PDMS diaphragm may be sealed in place, for example, by plasma activation of the surface and covalent bonding. That is, a portion of the surface of the PDMS diaphragm and a portion of the surface of the PDMS reservoir may be oxidized to remove some of the methyl groups, and expose the PDMS backbone containing hydroxyl groups. The oxidized surfaces may then be brought into contact to form a covalent Si—O—Si bond. Alternatively, PDMS prepolymer can be used to seal the PDMS diaphragm to the reservoir. For some embodiments of applicants' teachings, the diaphragm 28 is part of the micro-fluidic pump 14 (see FIG. 4) as will hereinafter be explained.

The micro-fluidic pump 14 has a housing 30 (see FIG. 4) that, in accordance with some embodiments of applicants' teachings can be manufactured similar to reservoir 18 of, for example, but not limited to, PDMS or other suitable biocompatible elastomer. Moreover, in accordance with some embodiments of applicants' teachings, the micro-fluidic pump 14 can deliver the substance, such as drug 26, at controlled flow rates, in the range of, for example, but not limited to, nL to μl/min. Illustrative, but not limiting, pressure generation can be in the range of 1-10 kPa.

Micro-fluidic pump 14 also has, in accordance with some embodiments of applicants' teachings, an on/off capability of a desired response time. Furthermore, the micro-fluidic pump 14 can have the capability to be operated remotely through, for example, but not limited to, inductive coupling, once implanted into the targeted area to allow for sustained dosing purposes.

In accordance with the various embodiments of applicants' teachings, micro-fluidic pump 14 operates by electro-osmosis. An electro-osmosis micro-fluidic pump 14 operates on an interfacial electro-osmotic phenomena, is electrically controllable, and allows for control of flow rate and desired on / off capability. Low-voltage, for example, 1-3 V, and low current, for example, 1-100 nA, can be applied to electro-osmosis micro-fluidic pumps to generate pressures in the range of 10 kPa. Moreover, electro-osmosis micro-fluidic pumps can be active and can be switched on at the time of choosing of an external controller. If desired, the flow rates of the electro-osmosis micro-fluidic pump 14 can be dynamically changed during the course of operation of the device. For example, the applied voltage can be modified in order to control the flow rate.

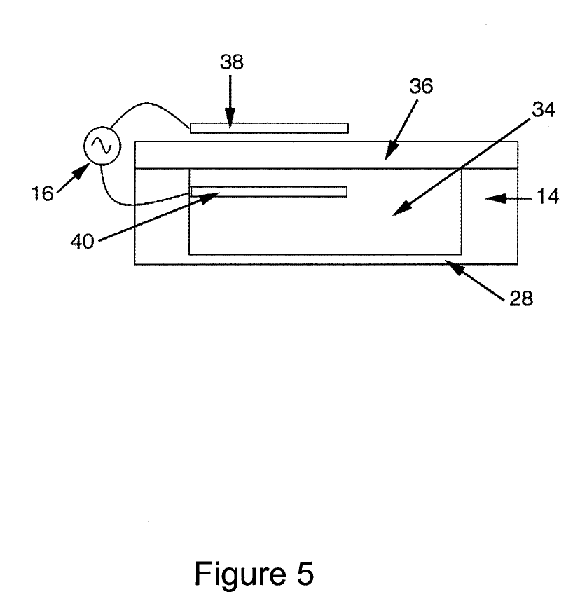

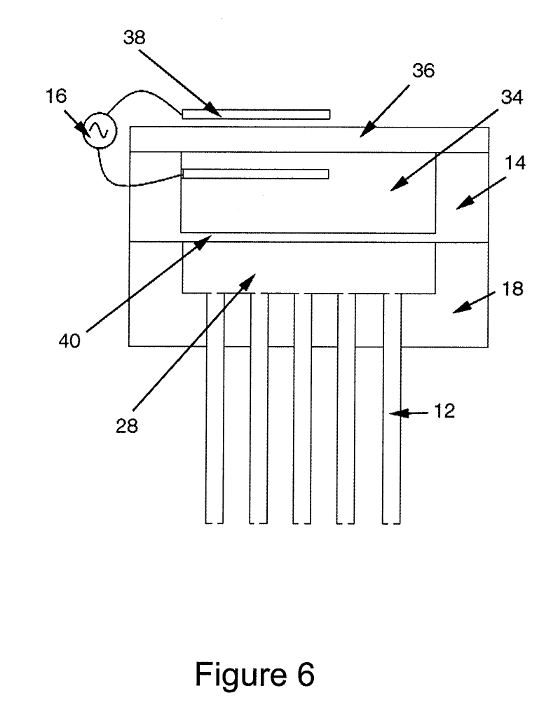

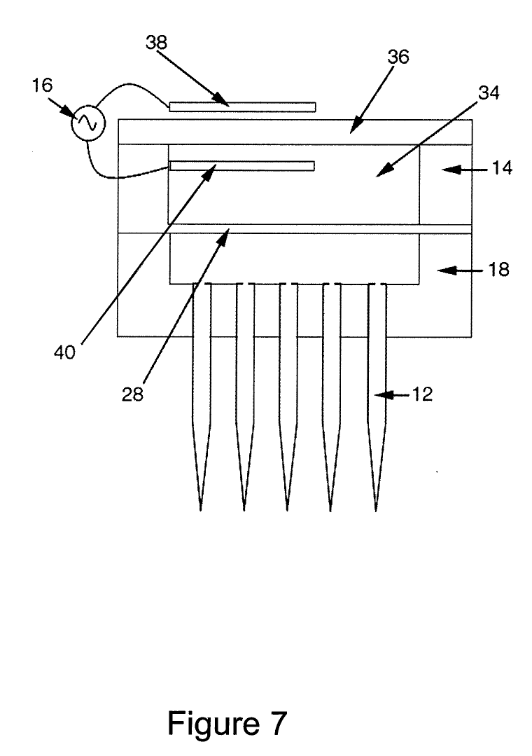

Referring to FIGS. 4-7, main chamber 32 of the micro-fluidic pump 14 is filled with, for example, but not limited to, salt 34 or other fluid absorbing material. Any salt (NaCl, KCl, MgCl2, CaCO3, etc.) or other substances, such as sugar, that dissolve in water may be used. The amount of fluid absorbing material used may depend on the particular embodiment. In some embodiments, main chamber 32 may be filled with between about 1 mm³ and about 5 mm³ of fluid absorbing material. Chamber 32 is then encased, in accordance with some embodiments of applicants' teachings in a semi-permeable membrane 36 that allows fluid to pass through it to access the salt 34. Suitable materials for the semi-permeable membrane 36 can include, for example, but not limited to, cellulose acetate, cationic and anionic selective membranes and metallized poly carbonate membranes. In some embodiments, the fluid that passes through membrane 36 to access the salt is extra cellular water present in or adjacent the tissue being treated. In other embodiments (not shown), an additional chamber may provided adjacent the main chamber 32 and may be filled with water such that the water may pass through membrane 36.

Without being limited by theory, it is believed that the basic principle of operation is due to the generation of an electrical double layer at the solid liquid interface (i.e. the interface of the fluid absorbing material, and the fluid). This introduces a relatively thin (10-100 nm) surface charge layer close to the walls of the micro-channels (i.e. the walls of the pores of the semi-permeable membrane). An electric field applied along the channel will drag the charged layer, which subsequently drags the bulk of the fluid through viscous drag. The force on the fluid is proportional to the electric field so that the amount of fluid transferred is proportional to the duration of operation of the pump.

The source 16 to produce a zero average current is then connected to the micro-fluidic pump 14 by having electrodes 38 and 40 positioned on either side of the semi-permeable membrane 36. The zero average current can include symmetrical currents such as symmetrical AC currents, or asymmetrical currents in which the average current over the period of the signal is zero. For example, an asymmetrical zero average current can be produced by applying a +10 mA current for 5 seconds and then a −5 mA current for 10 seconds. Since the amplitude and time period are different for the two halves of the cycle, there is asymmetry about the crossover point but the average current is zero (i.e., +10×5−5×10=0).

This is in contrast to a conventional electroosmotic/electrokinetic pump, in which a constant DC voltage is applied to the electrodes. With a conventional pump, if the voltage applied exceeds 1.2 V, hydrolysis occurs, generating hydrogen at the cathode and oxygen at the anode. This can be dangerous to health as well as inhibit the functioning of the pump. When a zero average current, such as a symmetrical AC current, is applied, however, the electrodes have one charge in the positive cycle and an opposite charge in the negative cycle. The reaction that takes place in the positive cycle is reversed in the negative cycle. Hence, no net reaction occurs at the electrodes and there is no gas evolution.

When a zero average current is applied, the electric field applied to the liquid switches direction from positive to negative as the voltage moves from the positive to the negative half of the cycle. The electroosmotic/electrokinetic flow is proportional to the magnitude and direction of the electric field in the solution and hence switches direction as well. The net average of the flow is therefore zero over the entire cycle for a zero average current application assuming that the resistance to flow is the same on both sides. However, the presence of salt on one side of the porous membrane changes the resistance to flow. Salt dissolves in the liquid, retaining it and increasing the resistance to flow back. This achieves rectification and directional flow even upon application of a zero average current.

In accordance with the various embodiments of applicants' teachings, the zero average current can be provided in a number of ways, for example, but not limited to direct connection to a power source generating current or voltage waveform, battery power with appropriate electronics for generation of DC power into an AC electric signal and inductive coupling of the AC signals to the micro-fluidic pump 14 from an external power source through use of, for example, but not limited to, a micro-coil attached to the electrodes.

Other methods, according to applicants' teachings can include, for example, but not limited to, using AC electric fields and some form of rectification to achieve uni-directional flow without generation of gas bubbles, using, for example, but not limited to, a third gate electrode apart from the two drive electrodes to modify the zeta-potential out of phase with the pumping signal in order to achieve rectification.

Referring to FIGS. 8 and 9, use of the delivery apparatus 10 can be seen. Apparatus 10 can be embedded within the target area, such as for example, the posterior of an eye (not illustrated). Once embedded, the source 16 of the AC signal can be switched on, allowing rectified flow 42 into the chamber 32, since the presence of salt 34 in the chamber 32 helps retain flow in the positive half of the AC cycle, and restrains backflow 44 in the negative half of the cycle.

The flow 42 swells the contents of chamber 32 causing diaphragm 28 to be deflected. This in turn pushes drug 26 out through the micro-needles 12 and into the tissue of the targeted area.

While the applicant's teachings are described in conjunction with various embodiments, it is not intended that the applicant's teachings be limited to such embodiments. On the contrary, the applicant's teachings encompass various alternatives, modifications, and equivalents, as will be appreciated by those of skill in the art.

1. A micro-pump comprising:
a housing defining at least a pumping chamber and a reservoir chamber, the pumping chamber and the reservoir chamber separated by a flexible diaphragm, the pumping chamber containing at least a fluid-absorbing material, and the pumping chamber having a semi-permeable membrane to allow fluid flow into and out of the pumping chamber;
at least two electrodes located on opposite sides of the semi-permeable membrane with at least one of the electrodes located in the pumping chamber; and
a source connected to the at least two electrodes capable of supplying a zero average current, so that supplying the zero average current to the at least two electrodes causes in combination with the fluid absorbing material in the pumping chamber a net flow of fluid across the semi-permeable membrane and into the pumping chamber to deflect the flexible diaphragm.

2. The micro-pump of claim 1, wherein the reservoir chamber contains a substance.

3. The micro-pump of claim 2, wherein the reservoir chamber comprises a delivery means for delivering the substance.

4. The micro-pump of claim 3, wherein the delivery means comprises one or more needles.

5. The micro-pump of claim 4, wherein the diaphragm deflects into the reservoir to displace the substance through the one or more needles.

6. The micro-pump of claim 1, wherein the zero average current is a symmetrical AC current.

7. The micro-pump of claim 1, wherein the fluid absorbing material is a salt.

8. The micro-pump of claim 1, wherein the housing is constructed of polydimethyl siloxane.

9. A substance delivery apparatus for delivering a substance to the posterior of an eye comprising:
a housing defining a reservoir containing the substance;
at least one micro-needle operably connected to the reservoir suitable for insertion into the posterior of the eye; and
a micro-pump fluidically connected to the reservoir so that when the micro-pump is activated, the substance is directed from the reservoir, through the at least one micro-needle.

10. The substance delivery apparatus of claim 9, wherein the micro-pump is operable to direct the substance from the reservoir at controlled flow rates.

11. The substance delivery apparatus of claim 9, wherein the micro-pump is remotely controllable.

12. The substance delivery apparatus of claim 9, wherein the micro-pump is an electro-osmosis micro-fluidic pump.

13. The substance delivery apparatus of claim 9, further comprising a flexible diaphragm between the micro-pump and the reservoir so that, when the micro-pump is activated, the flexible diaphragm is deflected into the reservoir.

14. The substance delivery apparatus of claim 9, wherein the at least one micro-needles are out of plane needles.

15. A method of constructing a substance delivery apparatus, the method comprising:
creating a mold of a housing defining a reservoir and one or more channels running from the reservoir to an outside edge of the housing;
casting the housing using the mold;
inserting a first end of one or more capillary tubes into the one or more channels;
shaping a second end of the one or more capillary tubes to form a micro-needle; and
loading a substance to be delivered into the reservoir.

16. The method of claim 15, wherein the shaping of the second end of the one or more capillary tubes comprises using a pipette puller to melt the second end and pull it to obtain a sharp tip.

17. The method of claim 16, wherein the shaping of the second end of the one or more capillary tubes occurs before the inserting of the first end of the one or more capillary tubes and the shaping of the second end of the one or more capillary tubes further comprises subjecting the first end of the one or more capillary tubes to plasma oxidation.

18. The method of claim 15, wherein the shaping of the second end of the one or more capillary tubes comprises extracting the second end from an enchant solution at a controlled rate.

19. The method of claim 15, wherein the shaping of the second end occurs after the inserting of the first end of the one or more capillary tubes.

20. The method of claim 15, wherein the reservoir is reloadable.

21. The method of claim 15, wherein the mold is created using a multilayer photolithography process.
